(19) United States
(12) Patent Application Publication
(10) Pub. No.: US 2018/0112205 A1
(43) Pub. Date: Apr. 26, 2018

(54) IN VITRO METHOD FOR STEM CELL PROLIFERATION AND USE OF A DEVICE FOR INCREASING THE PROLIFERATION OF STEM CELLS IN VITRO

(71) Applicant: INDIBA, S.A., SAINT QUIRZE DEL VALLES (BARCELONA) (ES)

(72) Inventors: Alejandro Ubeda Maeso, Madrid (ES); Maria Luisa Hernandez Bule, Madrid (ES)

(21) Appl. No.: 15/572,424

(22) PCT Filed: May 8, 2015
(86) PCT No.: PCT/ES2015/070372

§ 371 Date: Nov. 7, 2017

(51) Int. Cl. C12N 13/00; C12N 5/0775

(52) U.S. Cl. CPC C12N 13/00; C12N 5/0667

(57) ABSTRACT

In vitro method for stem cell proliferation and use of a device for increasing the proliferation of stem cells in vitro

The invention relates to an in vitro method for the proliferation of stem cells, comprising a step in which the stem cell culture is treated with an alternating current with a frequency in the 0.4 MHz to 0.6 MHz range, and to the use of a device for generating alternating current with a frequency in the 0.4 MHz to 0.6 MHz range in order to increase the proliferation of stem cells in vitro.

The present invention relates to the cell biology sector, specifically, to a method of proliferating stem cells carried out in vitro and to a device for increasing the proliferation of stem cells in vitro.

Electrotherapy based on electrothermal technology known as Capacitive and Resistive Electric Transfer (CRet) consists in a non-invasive strategy based on the use of alternating currents having frequencies in the range of 0.4 MHz to 0.6 MHz (a range comprised within the radiofrequency spectrum) in order to raise the temperature of the organs or tissues that are the target of the treatment by the action of said alternating electric currents. This type of technology has been shown to be effective in treatments in rehabilitation, regenerative and aesthetic medicine, for example, for the regeneration of lesions caused by trauma or degenerative lesions of the tissues, by reducing the associated pain, reducing inflammation, increasing the circulation of blood, improving vascular and muscular tone and improving the reabsorption of haematomas, oedemas and fluid accumulated in the joints and soft tissues.

In recent years, electrotherapy has begun to be used in the field of oncology in combination with chemotherapy or radiotherapy, an increase in the survival of patients treated with one of said combined therapies being shown.

At first, it was thought that the effects of electrotherapy resulted exclusively from the thermal component thereof and it was not recognised that the electric component could have a cellular effect.

However, in recent years, numerous studies and research have been conducted based on the use of electrotherapy in sub-thermal conditions with the aim of analysing the effect of the electric component on the cells and ruling out possible risks of oncogenesis or tumour development. Said studies have demonstrated that the electric component of the therapy does actually have an effect on the cells such that, when applied to cultures of tumour cell lines, cytostatic or cytotoxic effects were observed. Specifically, it has been demonstrated that:
An alternating current having a frequency of 0.57 MHz applied in sub-thermal conditions (5-minute pulses at 0.57 MHz with a current density of 50 μA/mm², applied every 4 hours for 12 to 24 hours) to cell cultures of the HepG2 cell line (filed under ATCC no. HB-8065) derived from human hepatocarcinoma cells, produces a cytostatic and cell differentiation effect thereon (Hernández-Bule, M. L. et al., International Journal of Oncology, 2007, 30, 583-592; Hernández-Bule, M. L. et al., International Journal of Oncology, 2010, 37, 1399-1405; and Hernández-Bule, M. L. et al., PLoS ONE, 2014, 9, 1e84636).
An alternating current having a frequency of 0.57 MHz applied in sub-thermal conditions (5-minute pulses at 0.57 MHz with a current density of 50 μA/mm², applied every 4 hours for 12 to 24 hours) to cell cultures of cell line NB69 (catalogue no. 99072802 Sigma in Sigma-Aldrich) derived from human neuroblastoma cells, produces a cytotoxic effect (Hernández-Bule, M. L. et al., International Journal of Oncology, 2012, 41, 1251-1259).

In addition to said anti-tumour effects, it has also been disclosed that an alternating current having a frequency of 0.57 MHz applied in sub-thermal conditions (5-minute pulses at 0.57 MHz with a current density of 50 μA/mm², applied every 4 hours for 12 to 24 hours) does not produce any detectable effect in cultures of peripheral blood mononuclear cells from healthy donors given that no change was observed in survival, necrosis and in the distribution of sub-populations of cells following treatment using said alternating electric current (Hernández-Bule, M. L. et al., International Journal of Oncology, 2012, 41, 1251-1259).

The inventors of this patent, following extensive and exhaustive experiments, have discovered surprisingly that the application of alternating electric currents having a frequency in the range of 0.4 to 0.6 MHz in cultures of stem cells in vitro increases the proliferation thereof without affecting the differentiation capacity thereof.

As used in the present document, ‘stem cell’ and the plural thereof refer to undifferentiated cells having the potential to be converted into one or more different cell types.

As used in the present document, ‘increase the proliferation’ refers to the generation of a larger number of cells in a particular period of time compared with the control cultures.

Thus, in a first aspect, the present invention relates to a method of proliferating stem cells in vitro characterised in that it comprises a step of treating the stem cell culture using alternating current having a frequency in the range of 0.4 MHz to 0.6 MHz.

It is envisaged that said stem cells may be of any type known in the prior art. Said stem cells may be totipotent, pluripotent or multipotent. In a preferred embodiment, the stem cells used in the proliferation method of the present invention are selected from the group which comprises multipotent stem cells, more preferably the stem cells used in the method of the present invention are multipotent mesenchymal stem cells.

As is known in the prior art, said mesenchymal stem cells may derive from various tissues. For the method of the present invention, said mesenchymal stem cells derive or are obtained preferably from adipose tissue, still more preferably, from subcutaneous adipose tissue. In the latter case, said stem cells used preferably have a passage of between 3 and 6.

The stem cells used may derive from any species of living being, more preferably said stem cells are mammalian stem cells and, still more preferably, said stem cells are human stem cells. Thus, in the most preferred embodiment, in the method of the present invention human mesenchymal stem cells are used, more preferably derived from subcutaneous adipose tissue.

Methods are known in the prior art for isolating the different stem cells from the various tissues from which said stem cells derive.

In a preferred embodiment, the alternating current has a frequency in the range of 0.4 MHz to 0.5 MHz, still more preferably, said alternating current has a frequency of 0.448 MHz.

The conditions for the above-mentioned treatment step in relation to the treatment time and/or the current density used depend on multiple factors, such as the size of the culture, the density of the culture and the separation between electrodes, among others. On the basis of said information, a person skilled in the art will be able to establish the appropriate treatment time and current density conditions.

It is envisaged that said treatment time and current density conditions may be established such that the treatment step is carried out in thermal conditions, that is, that in addition to the typical electric effect of the current applied, said current also causes an increase in temperature.

However, in a preferred embodiment, the treatment time and current density conditions are established so that the treatment step is carried out in sub-thermal conditions (that is, that only the typical electric effect of the current applied is produced without causing an increase in temperature). Preferably, the treatment step lasts between 12 and 72 hours, more preferably 48 hours. In addition, in said treatment step the current is applied preferably in pulses of current lasting between 1 and 10 minutes, more preferably 5 minutes, and separated by rests (period during which no current is applied) of between 1 and 8 hours, more preferably 4 hours. In this embodiment, the density of the alternating current is, preferably, between 1 and 100 μA/mm², still more preferably 50 μA/mm².

Thus, in the most preferred embodiment, the treatment step lasts for 48 hours and in said step, the current is applied at a density of 50 μA/mm² and in pulses lasting 5 minutes and separated by rests lasting 4 hours.

The treatment step of the in vitro proliferation method of the present invention is carried out using a pair of electrodes made of a suitable material (for example, stainless steel) applied to the culture and connected to an alternating current generating device. Preferably, the connection of the electrodes to the alternating current generating device is in series or parallel. More preferably, the connection of the electrodes is in series. Examples of alternating current generating devices are the following devices from Indiba®: Activ 902 model and ELITE model.

It is envisaged that the method of the present invention is carried out in any type of vessel or bioreactor available in the prior art for the culture of stem cells. In a preferred embodiment, the method is carried out in a Petri dish, still more preferably in a Petri dish 60 mm in diameter.

The method of the present invention also comprises one or more steps from among those commonly used in stem cell culture, such as counting the cells to be seeded, seeding a particular and appropriate number of cells, maintaining said cells in culture (with the corresponding changes of medium when the person skilled in the art considers it opportune or appropriate) and harvesting the cells (physical and/or enzymatic steps to obtain the cells, such as scraping and/or trypsinisation). In a preferred embodiment, the in vitro stem cell proliferation method of the present invention is characterised in that it comprises the steps of:

a) seeding the stem cells at a density of between 725 cells/cm² and 1360 cells/cm²;
b) treating the stem cell culture using alternating current having a frequency in the range of 0.4 MHz to 0.6 MHz;
c) maintaining the stem cells in culture; and
d) harvesting the stem cells.

The cell density range given in step a) of the method of the present invention refers to cells that grow in an adhesive manner (preferably human mesenchymal stem cells and, still more preferably, human mesenchymal stem cells derived from subcutaneous adipose tissue). However, a person skilled in the art will be able to extrapolate or adapt said density to the case of stem cells growing in suspension.

As mentioned earlier, it is envisaged that the method of the present invention may be carried out on any type of vessel or bioreactor available in the prior art for the culture of stem cells, more preferably, in a Petri dish, still more preferably, in a Petri dish 60 mm in diameter.

Step b) of treating the stem cell culture using alternating current having a frequency in the range of 0.4 MHz to 0.6 MHz, is as explained above.

In step c) of maintaining stem cells in culture, said stem cells are treated in accordance with the standard protocols known in the prior art and depending on the use that will be made of said cells. For the analysis of cell proliferation, the stem cells are harvested and/or processed directly on the cover glasses immediately after the final 5-minute treatment cycle. For studies of chondrogenic, adipogenic or osteogenic differentiation in mesenchymal stem cells, before the processing thereof, said stem cells are kept for 14 days in the respective differentiation mediums.

In step d) it is envisaged that, for harvesting the stem cells, physical, chemical or enzymatic means or combinations thereof are used. For example, when the stem cells grow adhering to a surface, to obtain the cells it is possible to combine enzymatic means, such as trypsinisation, with physical means, such as scraping.

The method of the present invention allows the proliferation of the cultured stem cells to be increased without affecting the capacity thereof for differentiation. Thus, it is envisaged that following the above-mentioned steps a stem cell differentiation step may be added or carried out. The characteristics of said cell differentiation step will depend on the type of stem cells used, which will have a specific differentiation potentiality or capacity.

Specifically, when human mesenchymal stem cells (preferably derived from subcutaneous adipose tissue) are used in the method of the present invention, the above-mentioned differentiation step may be, among others, an adipogenic, chondrogenic or osteogenic differentiation step so that said stem cells are differentiated into adipocytes, chondrocytes or osteocytes, respectively.

The culturing conditions that promote the differentiation of the different stem cells into the various cell types are known in the prior art. For example, for human mesenchymal stem cells (preferably derived from subcutaneous adipose tissue), the conditions for carrying out the above-mentioned differentiation processes are:
adipogenic differentiation: culture in the presence of suitable concentrations of 3-isobutyl-1-methylxanthine, indomethacin, insulin and dexamethasone, preferably 3-isobutyl-1-methylxanthine at a concentration of 0.25 mM, indomethacin at a concentration of 200 μM, insulin at a concentration of 10 μg/ml and dexamethasone at a concentration of 1 μM.
chondrogenic differentiation: culture in the presence of suitable concentrations of ascorbic acid 2-phosphate, TGF-β1, insulin and dexamethasone, preferably ascorbic acid 2-phosphate at a concentration of 150 nM, TGF-β1 at a concentration of 10 ng/mL, insulin at a concentration of 10 μg/ml and dexamethasone at a concentration of 100 nM.
osteogenic differentiation: culture in the presence of suitable concentrations of Bone Morphogenetic Protein 2 (BMP-2), dexamethasone, ascorbic acid 2-phosphate and β-glycerophosphate, preferably Bone Morphogenetic Protein 2 (BMP-2) at a concentration of 10 ng/ml, ascorbic acid 2-phosphate at a concentration of 50 μM, β-glycerophosphate at a concentration of 10 mM and dexamethasone at a concentration of 100 nM.

In an additional aspect, the present invention also discloses the use of a device for generating an alternating current having a frequency in the range of 0.4 MHz to 0.6 MHz to increase the proliferation of stem cells in vitro.

The conditions of use of the alternating current generating device are as explained above. Thus, in the most preferred embodiment, the alternating current generating device is used at a frequency of 0.448 MHz.

Also preferably, said alternating current generating device is used for 48 hours, by the application of pulses of current lasting 5 minutes with rests of 4 hours between said pulses of current and at a current density of 50 μA/mm².

The stem cells are also as explained above. Thus, in the most preferred embodiment, said stem cells are human mesenchymal stem cells derived from subcutaneous adipose tissue, still more preferably of a passage of between 3 and 6.

To aid understanding, the present invention is described below in more detail with reference to the accompanying drawings, which are given as an example, and with reference to illustrative and non-limiting examples.

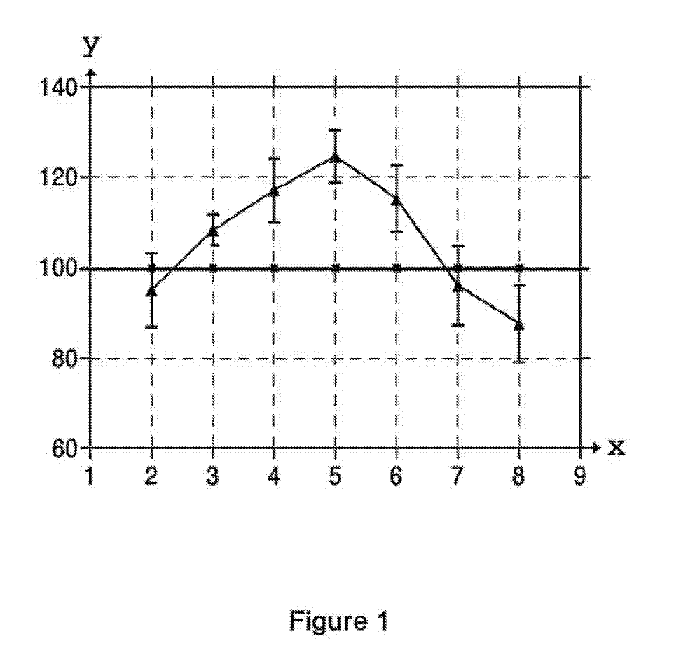

FIG. 1 shows the results of the fluorescence assay carried out in example 2 of the present invention. The triangles shown, and the graph said triangles describe, correspond to the human mesenchymal cell cultures derived from subcutaneous adipose tissue of passages 2 to 8 treated using alternating current having a frequency of 0.448 MHz. The results for the number of cells are shown expressed as a percentage of the number of cells obtained in the respective controls. In the figure, the ordinate axis (y) corresponds to the number of cells expressed as a percentage of the number of cells observed in the corresponding control; and the abscissa axis (x) corresponds to the passage number.

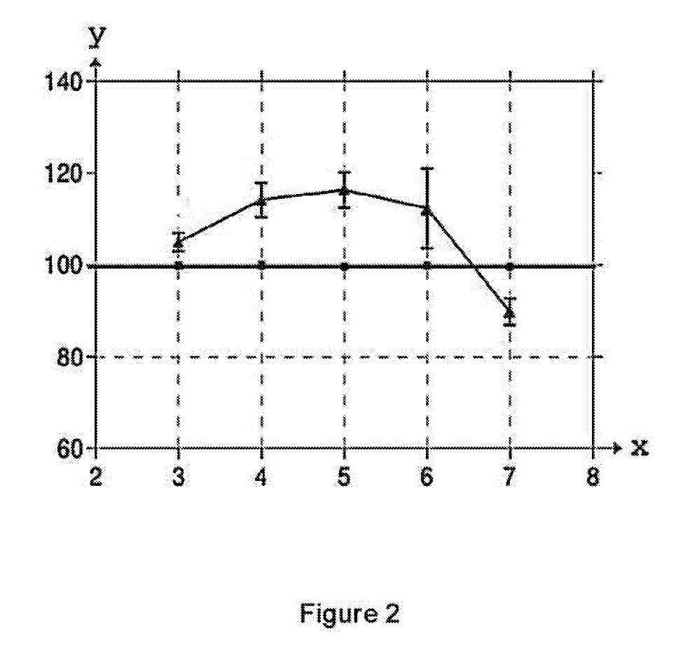

FIG. 2 shows the results obtained in the XTT assay carried out in example 3 of the present invention. The triangles shown, and the graph said triangles describe, correspond to the human mesenchymal cell cultures derived from subcutaneous adipose tissue of passages 3 to 7 treated using alternating current having a frequency of 0.448 MHz. The results show the measured absorbency at 492 nm in each of the passages as a percentage of that measured in the corresponding control. In the figure, the ordinate axis (y) corresponds to the measured absorbency at 492 nm as a percentage of the observed absorbency (at the same wavelength) in the corresponding control; and the abscissa axis (x) corresponds to the passage number.

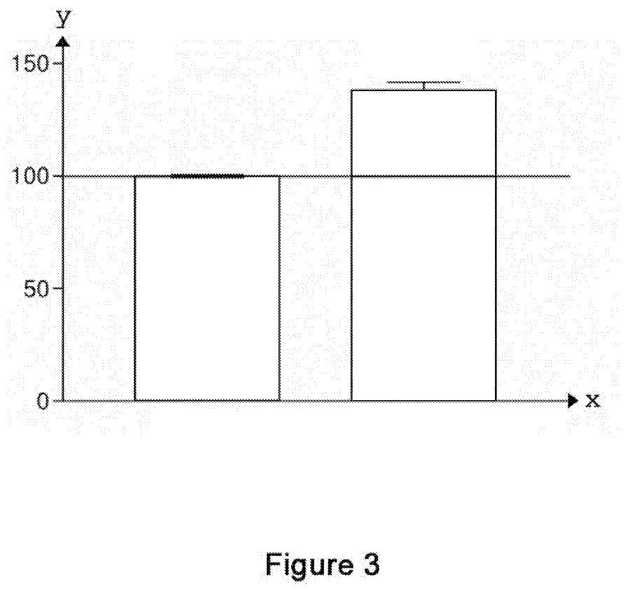

FIG. 3 shows the results obtained in the bromodeoxyuridine incorporation assay of example 4 of the present invention. The left-hand bar corresponds to the results obtained for the control cultures of mesenchymal cells derived from subcutaneous adipose tissue; and the right-hand bar corresponds to the results obtained for the human mesenchymal cell cultures derived from subcutaneous adipose tissue treated using alternating current having a frequency of 0.448 MHz. The results for the number of cells are shown expressed as a percentage of the number of cells obtained in the respective controls. In the figure, the ordinate axis (y) corresponds to the number of cells expressed as a percentage of the number of cells observed in the corresponding control; and the abscissa axis (x) corresponds, as mentioned above, to the group analysed.

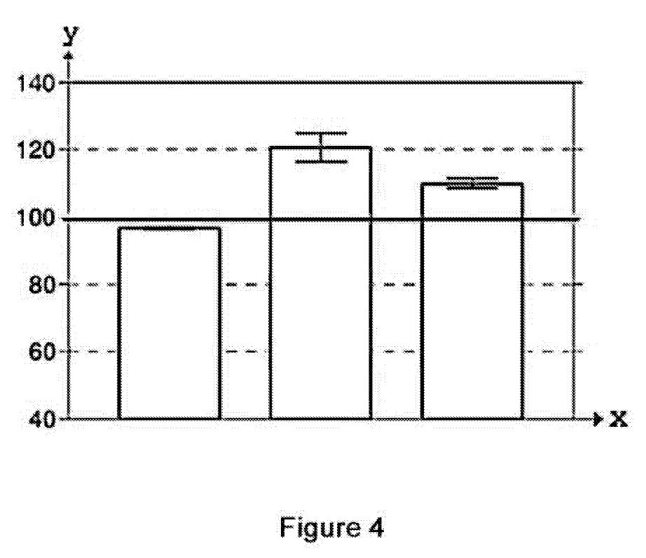

FIG. 4 shows the results of the analysis of the cell cycle obtained in example 5 of the present invention. All the bars shown correspond to the human mesenchymal cell cultures derived from subcutaneous adipose tissue treated using alternating current having a frequency of 0.448 MHz. The left-hand bar refers to the G0/G1 phase of the cell cycle. The central bar corresponds to the S phase of the cell cycle. The right-hand bar, meanwhile, refers to the G2/M phase of the cell cycle. The results are shown expressed as a percentage compared with the control. In the figure, the ordinate axis (y) corresponds to the percentage compared with the percentage observed in the control; and the abscissa axis (x) corresponds, as mentioned above, to the phases of the cell cycle.

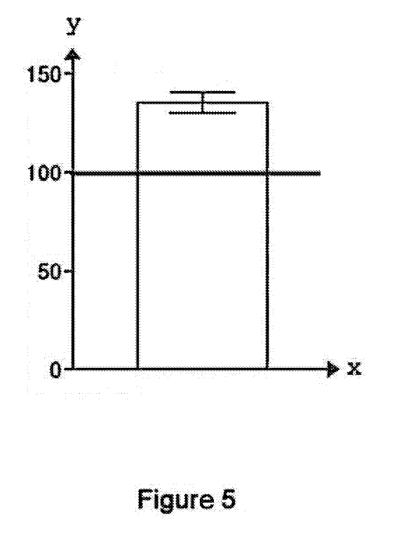

FIG. 5 shows the results obtained in the immunofluorescence assay relating to the expression of Proliferating Cell Nuclear Antigen (PCNA) for the human mesenchymal cell cultures derived from subcutaneous adipose tissue treated using alternating current having a frequency of 0.448 MHz, in example 6 of the present invention. The results are shown expressed as a percentage compared with the control. In the figure, the ordinate axis (y) corresponds to the percentage compared with the control; and the abscissa axis (x) corresponds, as mentioned above, to the group analysed.

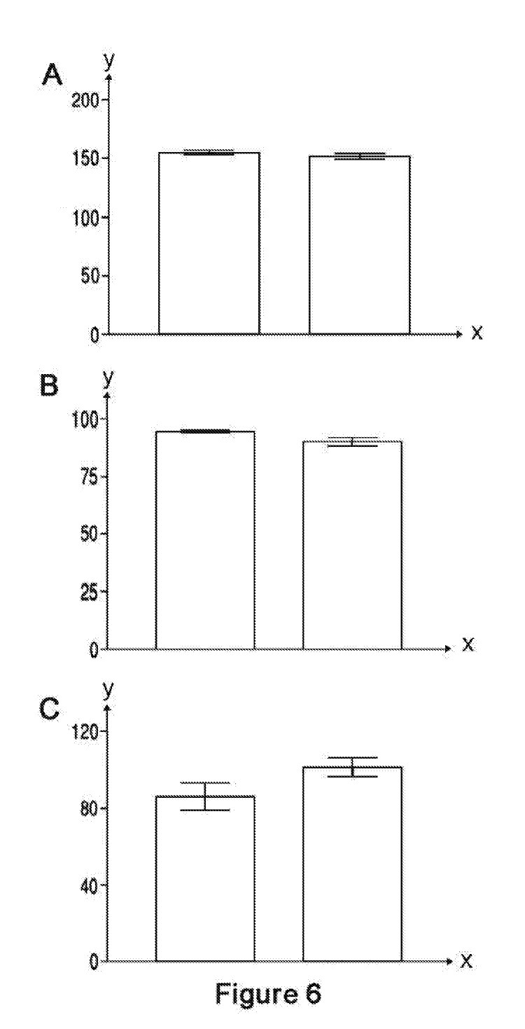

FIG. 6 shows the results obtained in the assays analysing differentiation capacity carried out in example 7 of the present invention. FIG. 6 A shows the results relating to adipogenic differentiation; FIG. 6 B shows the results relating to chondrogenic differentiation; and FIG. 6 C shows the results relating to osteogenic differentiation. In the three figures, the left-hand bar corresponds to the results obtained for the control cultures of mesenchymal cells derived from subcutaneous adipose tissue; and the right-hand bar corresponds to the results obtained for the human mesenchymal cell cultures derived from subcutaneous adipose tissue treated using alternating current having a frequency of 0.448 MHz. In the three figures the ordinate axis (y) refers to the average optical density and the abscissa axis (x), as mentioned above, refers to the group analysed. The optical density values were obtained by computer-assisted photomicrographic image analysis of the biological samples, using the AnalySIS 3.1 program.

### EXAMPLES

### Example 1. Obtaining Mesenchymal Stem Cells Derived from Subcutaneous Adipose Tissue

The mesenchymal stem cells derived from adipose tissue were isolated from samples of subcutaneous adipose tissue obtained during general surgical procedures, from four healthy donors (two men, aged 65 and 69 years, and two women, aged 29 and 35 years).

The study and procedures were evaluated and approved by the Clinical Research Ethics Committee of the Ramón y Cajal University Hospital (Madrid, Spain) and the volunteers agreed to the donation in writing by a standard informed consent procedure.

Pieces of adipose tissue measuring 0.5-1 cm³ obtained from the above-mentioned patients were cleaned so as to eliminate fibrotic tissue, visible fascia and blood vessels therefrom. Next, said pieces of adipose tissue were cut using a scalpel into small scraps or fragments measuring 1-2 mm³. The fragments were subjected to digestion using collagenase A at a concentration of 1 mg/ml (Roche Applied Science, Basel, Switzerland) in Hank's Balanced Salt Solution (HBSS) (Hyclone, Sur Logan, Utah, USA) for 40 minutes at 37° C. while stirring gently.

The collagenase A activity was stopped using foetal bovine serum in DMEM culture medium having a high glucose concentration (Biowhittaker, Verviers, Belgium).

Next, the cells present in the fragments were detached using a P1000 micropipette and MultiGuard tips (Sorenson BioScience, Salt Lake City, Utah, USA). Once detached, the large pieces of tissue and/or the non-detached blood vessels could be precipitated for 2 minutes to the bottom of a sterile centrifuge tube and the cell suspension (supernatant) harvested.

The cell suspension collected was transferred to a different tube and centrifuged at 300 G for 5 minutes to produce the stromal vascular fraction of the cells by sedimentation. After aspirating the supernatant and the floating layer of adipose cells, the sediment or pellet was re-suspended in MesenPro culture medium (MesenPro-RS®, Gibco, Invitrogen, Camarillo, Calif., USA) supplemented with 1% glutamine (Gibco, Invitrogen, Camarillo, Calif., USA) and 1% penicillin-streptomycin (Gibco, Invitrogen, Camarillo, Calif., USA) and seeded in a 75 cm² T-flask (Falcon, Corning, USA). After 48 hours, the culture was washed twice using Hank's balanced salt solution to remove any residue and cells not attached to the flask. After the washes, MesenPro medium was again administered to the cell culture, as indicated above. Two days later, on the fourth day, the medium was replaced. On the seventh day, when the cells were confluent, said cells were passaged. To do this, the cells were detached from the flask using 0.05% trypsin and 0.02% EDTA (Sigma, Saint Louis, Mo., USA) in Hank's balanced salt solution. Once obtained, the cells were seeded in a new dish at a density of 670 cells/cm². When the culture reached confluence, the cells were harvested as indicated above, divided into aliquots and said aliquots were frozen in a solution consisting of 10% by volume DMSO (Sigma, Saint Louis, Mo., USA) and 90% by volume of foetal bovine serum (Gibco, Invitrogen, Paisley, Scotland, United Kingdom).

### Example 2. Analysis by Fluorescent Staining of the Cell Nuclei of the Increase in the Proliferation of Human Mesenchymal Stem Cells Derived from Subcutaneous Adipose Tissue after Treatment with Alternating Current

From the stem cells obtained in accordance with example 1, five Petri dishes were cultured for treatment with alternating current and five control Petri dishes for each of the following conditions: cells from passage 2, from passage 3, from passage 4, from passage 5, from passage 6, from passage 7 and from passage 8, giving a total of 70 Petri dishes. In all cases, the Petri dishes used were 60 mm in diameter.

The culture density of the cells was 725 cells/cm². Four days after seeding, said dishes were treated with alternating current using pairs of stainless steel electrodes as described in the prior art (see, for example, Hernández-Bule, M. L. et al., International Journal of Oncology, 2007, 30, 583-592), connected in series to the Indiba Activ 902 alternating current generating device (INDIBA®, Barcelona, Spain). The treatment lasted for 48 hours and the device was configured to a frequency of 0.448 MHz and such that the treatment pattern was as follows: 5-minute pulses of current followed by 4-hour rests, with a current density of 50 μA/mm².

After 48 hours of treatment, the cells were fixed using 4% paraformaldehyde, permeabilised using 0.1% Triton in Phosphate Buffered Saline (PBS) and the cell nuclei were stained using bisBenzimide H 33258 (Sigma, Saint Louis, Mo., USA) at a concentration of 10⁻⁵ M.

Next, the number of cells on the surface of the Petri dishes was counted, specifically on the surface located within the rectangle delimited by the two electrodes used for the treatment.

The cell count was carried out using an Olympus IX-70 fluorescence microscope. A 10× objective lens was used and 24 randomly selected microscopic fields (840 μm×630 μm; area=0.5292 mm²) separated by an average distance of 1.25 mm were counted selected randomly, and were photographed and analysed. Nuclei with at least half their area included within a field were counted and the total number of cells in the area treated was estimated from said count.

The results obtained are summarised in FIG. 1. In said figure, an increase of between 5% and 25% in the quantity of cells in passages 3 to 6 can be seen when the culture of human mesenchymal cells derived from subcutaneous adipose tissue was treated using alternating current as indicated above.

### Example 3. Analysis by XTT Assay of the Increase in the Proliferation of Human Mesenchymal Stem Cells Derived from Subcutaneous Adipose Tissue after Treatment with Alternating Current

The results of example 2 were corroborated by carrying out the corresponding experiment to measure the proliferation by XTT assay. To do this, the cultures were produced as explained in said example 2, but on this occasion, passages 3 to 7 were analysed.

Following the treatment step, the cultured cells inside the treatment area (between the electrodes), for both the control group and the treated group, were incubated with XTT tetrazolium salt for 3 hours at a temperature of 37° C. and an atmosphere of 6.5% CO₂. The metabolically active cells reduced the XTT salt producing intensely coloured formazan compounds, which were quantified using a microplate reader (TECAN, Mannedorf, Switzerland) at a wavelength of 492 nm. The values obtained correlated directly with the number of active cells.

The results obtained are summarised in FIG. 2. Consistent with the results obtained by counting the cells using fluorescent staining of the nuclei, an increase of approximately 5% to 25% in absorbency in passages 3 to 6 was also demonstrated using the XTT assay when the culture of human mesenchymal cells derived from subcutaneous adipose tissue was treated using alternating current. Said increase in absorbency is due to an increase in the number of cells (increase in proliferation).

### Example 4. Analysis by Bromodeoxyuridine Incorporation Assay of the Increase in the Proliferation of Human Mesenchymal Stem Cells Derived from Subcutaneous Adipose Tissue after Treatment with Alternating Current

The proliferation of the mesenchymal cells of example 1 was also analysed by bromodeoxyuridine incorporation assay. In this case, cells were analysed from passages 3 to 5 which were cultured in Petri dishes in 12 mm cover glasses placed in the zone located between the electrodes. The culture and the treatment of the cells using alternating current were carried out as indicated in example 2, the only change being that in the last six hours of treatment the cultures were incubated in the presence of bromodeoxyuridine at a concentration of 3 mM.

After treatment with alternating current, the cells were fixed using 4% paraformaldehyde and permeabilised using ethanol:acetic acid (95:5) for 10 minutes at 4° C. The cells in the cover glasses were incubated overnight at 4° C. with anti-BrdU mouse monoclonal antibody (dilution 1:20, Dako, Glostrup, Denmark), followed by one hour of immunofluorescent marking by incubation at ambient temperature with anti-mouse IgG antibody combined with Alexa Fluor 568 (dilution 1:500, Molecular Probes, Invitrogen, Camarillo, Calif., USA). The cell nuclei were fluorescently stained using bisBenzimide H 33258 (Sigma, Saint Louis, Mo., USA), as indicated previously in example 2, except for the fact that in the present procedure the cells were processed on the cover glasses in which said cells had grown.

The cover glasses were analysed in a Nikon Eclipse TE300 fluorescence microscope. Four experimental repetitions were carried out, each with four cover glasses per experimental group. The number of total nuclei and of bromodeoxyuridine-positive cells was counted in the selected fields by systematic random sampling. A total of 15 fields per cover glass were studied. The images were recorded and analysed using AnalySIS 3.1 software (Soft Imaging Systems GmbH, Münster, Germany).

The results obtained are summarised in FIG. 3. The quantity of proliferating cells present in the different cultures (treated or control) was analysed directly using this assay. As can be seen in FIG. 3, in the group of cultures to which the alternating current was applied (treated cultures), a significant increase of about 38% in the number of proliferating cells was observed compared with the controls.

### Example 5. Analysis of Cell Cycle Impairment Due to the Alternating Current Treatment Step

The cells isolated in accordance with example 1 were cultured and treated as indicated in example 2. In this case, only mesenchymal stem cells from passages 3 and 4 were used.

After the treatment step, the cells growing within the zone comprised between the electrodes were harvested using trypsin, placed in Eppendorf tubes and fixed by treatment in 1 ml of 70% ethanol at 4° C. overnight. The samples of approximately 1×10⁵ cells per dish were washed twice in Phosphate Buffered Saline and incubated for one hour in darkness at ambient temperature with propidium iodide staining solution at 20 mg/ml (Boehringer, Ingelheim, Germany) supplemented with RNasa A (200 ng/ml; Boehringer, Ingelheim, Germany) in citrate buffer at a concentration of 3.4 mM.

The cells were analysed in a flow cytometer (FACScalibur, BD Biosciences, San Jose, Calif., USA). Ten thousand events per sample were acquired using CellQuest software 3.2 (BD Biosciences).

The results obtained are summarised in FIG. 4, which shows that in the cell cultures treated using alternating current, there was an increase in the number of cells in the S and G2/M phases of the cell cycle and a slight reduction in the number of cells in the G0/G1 phase of the cell cycle, all the above in comparison with the controls. These results are consistent with those explained earlier and are indicative of an increase in cell proliferation (it was observed that the treatment stimulated the progression of the cells through the different phases of the cell cycle).

### Example 6. Analysis of the Variation in the Expression of Proliferating Cell Nuclear Antigen (PCNA) Due to the Alternating Current Treatment Step

The cells isolated according to example 1 were cultured and treated as indicated in example 2. In this case, only mesenchymal stem cells from passages 3 to 5 were used, seeded in cover glasses placed on the 60 mm diameter Petri dishes.

Proliferating Cell Nuclear Antigen (PCNA) is a protein associated with the DNA polymerase which is normally used as a marker for cells which are in the S and G2 phases of the cell cycle.

After the treatment step, the cells were fixed using 4% paraformaldehyde and were permeabilised in ethanol:acetic acid in a proportion of 95:5, incubated overnight at 4° C. in the presence of an anti-PCNA antibody (Santa Cruz Biotechnologies, TX, USA) and finally were fluorescently stained by incubation with anti-mouse IgG combined with Alexa Fluor 488 (Molecular Probes, Invitrogen, Camarillo, Calif., USA) for one hour at ambient temperature. The cell nuclei were counterstained using bisBenzimide H 33258 (Sigma, Saint Louis, Mo., USA).

The percentage of PCNA-positive cells in the different groups (control cultures and treated cultures) was estimated as described in example 4 for counting the number of total nuclei and of bromodeoxyuridine-positive cells.

The results obtained are shown in FIG. 5. In said figure, it can be seen that the human mesenchymal cell cultures treated using alternating current showed an increase of approximately 35% in the number of cells which expressed the PCNA marker compared with with the controls. These results showed an increase in proliferation in cultures of human mesenchymal cells treated using alternating current.

### Example 7. Study of the Differentiation Capacity of Human Mesenchymal Stem Cells Derived from Subcutaneous Adipose Tissue Treated Using Alternating Current

The initial culture and treatment of mesenchymal cells obtained in accordance with example 1 were carried out as indicated in example 2.

Following the treatment step, the dishes were kept for 14 days in the corresponding differentiator medium, three experimental repeats were carried out, each with four treated dishes and four control dishes per repeat and differentiator medium:
Study of adipogenic differentiation capacity: basal medium was used (DMEM having a high glucose concentration, 20% foetal bovine serum, 1% glutamine and 1% penicillin-streptomycin) to which 3-isobutyl-1-methylxanthine was added at a concentration of 0.25 mM, indomethacin at a concentration of 200 μM, insulin at a concentration of 10 μg/ml and dexamethasone at a concentration of 1 μM.
Study of chondrogenic differentiation capacity: basal medium was used (DMEM having a high glucose concentration, 20% foetal bovine serum, 1% glutamine and 1% penicillin-streptomycin) to which ascorbic acid 2-phosphate was added at a concentration of 150 nM, TGF-β1 at a concentration of 10 ng/mL, insulin at a concentration of 10 μg/ml and dexamethasone at a concentration of 100 nM.
Study of osteogenic differentiation capacity: basal medium was used (DMEM having a high glucose concentration, 20% foetal bovine serum, 1% glutamine and 1% penicillin-streptomycin) to which Bone Morphogenetic Protein 2 (BMP-2) was added at a concentration of 10 ng/ml, ascorbic acid 2-phosphate at a concentration of 50 μM, β-glycerophosphate at a concentration of 10 mM and dexamethasone at a concentration of 100 nM.

The same process was carried out in parallel for the respective controls.

Next, the cultures were fixed and stained using oil red O (Sigma, Saint Louis, Mo., USA) for the analysis of adipogenic differentiation, using alcian blue for the analysis of chondrogenic differentiation and using alizarin red for the analysis of osteogenic differentiation.

The results obtained with respect to the quantification of the staining are shown in FIG. 6.

FIG. 6 A shows that there was no difference in oil red O staining between the cultures treated using alternating current and the controls. It was therefore deduced that the treatment step using alternating current did not affect the adipogenic differentiation capacity of said human mesenchymal cells.

FIG. 6 B shows that there was no difference in alcian blue staining between the cultures treated using alternating current and the controls. It was therefore deduced that the treatment step using alternating current did not affect the chondrogenic differentiation capacity of said human mesenchymal cells.

FIG. 6 C shows that there was no difference in alizarin red staining between the cultures treated using alternating current and the controls. It was therefore deduced that the treatment step using alternating current did not affect the osteogenic differentiation capacity of said human mesenchymal cells.

Although the invention has been described with reference to preferred embodiments, said embodiments should not be considered as limiting the invention, which will be defined by the widest interpretation of the following claims.

1. In vitro method for proliferation of stem cells comprising treating the stem cell culture using alternating current having a frequency in the range of 0.4 MHz to 0.6 MHz.

2. Method according to claim 1, the stem cells are human mesenchymal stem cells.

3. Method according to claim 2, wherein the human mesenchymal stem cells are derived from subcutaneous adipose tissue.

4. Method according to claim 3, wherein the human mesenchymal stem cells derived from adipose tissue are from a passage of between 3 and 6.

5. Method according to claim 1, wherein the radiofrequency current has a frequency in the range of 0.4 MHz to 0.5 MHz.

6. Method according to claim 5, wherein the radiofrequency current has a frequency of 0.448 MHz.

7. Method according to claim 1, wherein the treatment lasts 48 hours.

8. Method according to claim 1, the treatment is applied in pulses lasting 5 minutes and separated by rests lasting 4 hours.

9. Method according to claim 1, wherein the current density of the radiofrequency current is 50 μA/mm².

10. In vitro method for proliferation of stem cells comprising:
a) seeding the stem cells at a density of between 725 cells/cm² and 1360 cells/cm²;
b) treating the stem cell culture using alternating current having a frequency in the range of 0.4 MHz to 0.6 MHz;
c) maintaining the stem cells in culture; and
d) harvesting the stem cells.

11-12. (canceled)

13. Method according to claim 1, wherein a radiofrequency generating device is used for 48 hours to apply pulses of current lasting 5 minutes with rests of 4 hours between said pulses of current with a current density of 50 μA/mm².

14. (canceled)

15. Method according to claim 10, wherein a radiofrequency generating device is used for 48 hours to apply pulses of current lasting 5 minutes with rests of 4 hours between said pulses of current with a current density of 50 μA/mm².
